(19) United States
(12) Patent Application Publication
(10) Pub. No.: US 2026/0137892 A1
(43) Pub. Date: May 21, 2026

(54) CPAP Bracket Device

(71) Applicant: Mathias Reidenbach, Cameron, NC (US)

(72) Inventor: Mathias Reidenbach, Cameron, NC (US)

(21) Appl. No.: 19/365,570

(22) Filed: Oct. 22, 2025

Related U.S. Application Data
(60) Provisional application No. 63/720,793, filed on Nov. 15, 2024.

(51) Int. Cl. A61M 16/06

(52) U.S. Cl. CPC A61M 16/0683; A61M 2205/58; A61M 2209/082

(57) ABSTRACT

A CPAP bracket device is disclosed, which allows elderly, handicapped, or other physically limited individuals to put on a CPAP mask without assistance. The device is comprised of an aluminum bracket component that may be mounted to a wall. The bracket component includes two bent aluminum rods which are used to hold the CPAP headgear and mask. The headgear and mask are held in such a way that the user can slide their head toward the mount and apply their headgear with ease. The mount also offers a secure storage place for the mask and headgear to prevent it from being misplaced, or to conveniently dry it after it has been washed.

### CROSS-REFERENCE TO RELATED APPLICATION

The present application claims priority to, and the benefit of, U.S. Provisional Application No. 63/720,793, which was filed on Nov. 15, 2024, and is incorporated herein by reference in its entirety.

### FIELD OF THE INVENTION

The present invention relates generally to the field of CPAP bracket devices. More specifically, the present invention relates to a wall-mounted aluminum bracket that supports CPAP headgear and its mask. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in CPAP bracket devices. Generally, people with sleep apnea may need a CPAP machine to get a healthy night's rest. Further, people with limited strength and dexterity, especially the elderly, may have trouble putting on their CPAP headgear and mask. However, being unable to use their CPAP nightly can lead to increased risk of stroke, especially for those with severe obstructive sleep apnea. Additionally, CPAP headgear can accumulate moisture and develop bacteria if not dried properly.

CPAP stands for continuous positive airway pressure. It is a machine that uses mild air pressure to keep breathing airways open while you sleep, such that a constant level of pressure greater than atmospheric pressure is continuously applied to the upper respiratory tract of a person. CPAP is used to treat sleep-related breathing disorders including sleep apnea. Specifically, the application of positive pressure may be intended to prevent upper airway collapse, as occurs in obstructive sleep apnea, or to reduce the work of breathing in conditions such as acute decompensated heart failure.

Accordingly, there is a demand for an improved CPAP bracket device that provides users with a wall-mounted bracket that supports CPAP headgear and its mask. More particularly, there is a demand for a CPAP bracket device that offers a way to dry out the mask and headgear after washing it to prevent the development of mold and bacteria.

Therefore, there exists a long felt need in the art for a CPAP bracket device that provides users with a wall-mounted bracket that supports CPAP headgear and its mask. There is also a long felt need in the art for a CPAP bracket device that features two bent aluminum rods that support the headgear and its straps in an upright position. Further, there is a long felt need in the art for a CPAP bracket device that allows users to keep their mask easily accessible and put it on without having to ask another person for assistance. Moreover, there is a long felt need in the art for a device that offers a way to dry out the mask and headgear after washing it to prevent the development of mold and bacteria. Further, there is a long felt need in the art for a CPAP bracket handle device that is comprised of aluminum. Finally, there is a long felt need in the art for a CPAP bracket device that retains the headgear and mask in such a way that the user can slide their head toward the mount and apply their headgear with ease.

The subject matter disclosed and claimed herein, in one embodiment thereof, comprises a CPAP bracket device. The device allows elderly, handicapped, or other physically limited individuals to put on a CPAP mask without assistance. The device is comprised of an aluminum bracket component that may be mounted to a wall. The bracket component includes two bent aluminum rods which are used to hold the CPAP headgear and mask. The headgear and mask are held in such a way that the user can slide their head toward the mount and apply their headgear with ease. The mount also offers a secure storage place for the mask and headgear to prevent it from being misplaced, or to conveniently dry it after it has been washed.

In this manner, the CPAP bracket device of the present invention accomplishes all of the forgoing objectives and provides users with a device that supports CPAP headgear and mask with ease. The device is an aluminum bracket mounted to a wall. The device includes aluminum rods to hold the CPAP headgear and mask.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a CPAP bracket device. The device is comprised of a bracket component that may be mounted to a wall. The bracket component includes two bent rods which are used to hold the CPAP headgear and mask. The device offers a secure storage place for the mask and headgear to prevent it from being misplaced.

In one embodiment, the CPAP bracket device offers a secure storage place for CPAP mask and headgear. Thus, the device allows users to keep their mask and headgear easily accessible and be able to put it on without having to ask another person for assistance. Further, the CPAP bracket device also offers a way to dry out the CPAP mask and headgear after washing it to prevent the development of mold and bacteria.

In one embodiment, the CPAP bracket device comprises a bracket component that may be mounted to a wall or other vertical support surface, as needed. The bracket component comprises an L-shaped support component that is secured directly to the wall or other vertical support surface. The L-shaped support component can be any suitable size and length as is known in the art, as long as the L-shaped support component is of a size and length to support a standard CPAP mask and headgear with ease. Further, the L-shaped support component comprises a front surface which faces out from the wall and a rear surface which secures to the wall. Typically, the L-shaped support component is secured to the wall via wall anchors, screws, nails, bolts, etc., or any other suitable fastening means as is known in the art.

In one embodiment, a pair of posts are secured near the middle of the front surface of the L-shaped support component via screws, nails, bolts, etc., or any suitable fastening means as is known in the art. The pair of posts are positioned perpendicular to the L-shaped support component. The pair of posts are secured with a front plate at the front to keep the pair of posts secured together during use.

In one embodiment, the bracket component includes two bent rods which are used to hold the CPAP headgear and mask. The two bent rods are secured to a top of the pair of posts and angle outwards and upwards. The two bent rods are secured via screws, nails, bolts, etc., or any other suitable fastening means as is known in the art. The two bent rods are angled in such a fashion that CPAP mask and headgear can be hung from the rods. Further, the ends of the two bent rods can comprise eyelets or other openings, which parts of the headgear or mask can be secured to.

In one embodiment, the CPAP bracket device allows elderly, handicapped, or other physically limited individuals to put on a CPAP mask without assistance. Specifically, the two bent rods are angled, such that the CPAP headgear and mask are held in such a way that the user can slide their head toward the mount and apply their headgear with ease. Thus, the CPAP bracket device allows users to keep their mask easily accessible and put it on without having to ask another person for assistance.

In one embodiment, the CPAP bracket device also offers a secure storage place for the mask and headgear to prevent it from being misplaced, or to conveniently dry it after it has been washed to prevent the development of mold and bacteria.

In one embodiment, the CPAP bracket device is manufactured from aluminum, stainless steel, plastic, such as polycarbonate, or other suitable materials as is known in the art, or a combination of such materials.

In yet another embodiment, the CPAP bracket device comprises a plurality of indicia.

In yet another embodiment, a method of supporting CPAP headgear and mask to keep the mask easily accessible is disclosed. The method includes the steps of providing a bracket component that may be mounted to a wall. The method also comprises mounting the bracket component to a wall. Further, the method comprises positioning the mask and headgear on the bracket component. The method also comprises sliding a user's head toward the mask and headgear, such that the mask is applied with ease. Finally, the method comprises preventing the mask and headgear from being misplaced, or to conveniently dry it after it has been washed.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the CPAP bracket device of the present invention in accordance with the disclosed architecture;

FIG. 2 illustrates a perspective view of one embodiment of the CPAP bracket device of the present invention showing the bracket component in accordance with the disclosed architecture;

FIG. 3 illustrates a perspective view of one embodiment of the CPAP bracket device of the present invention showing the device mounted to a wall in accordance with the disclosed architecture;

FIG. 4 illustrates a perspective view of one embodiment of the CPAP bracket device of the present invention showing the two bent aluminum rods to retain the mask in position in accordance with the disclosed architecture;

FIG. 5 illustrates a perspective view of one embodiment of the CPAP bracket device of the present invention showing the device in use in accordance with the disclosed architecture; and

FIG. 6 illustrates a flowchart showing the method of supporting CPAP headgear and mask to keep the mask easily accessible in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a CPAP bracket device that provides users with a wall-mounted bracket that supports CPAP headgear and its mask. There is also a long felt need in the art for a CPAP bracket device that features two bent aluminum rods that support the headgear and its straps in an upright position. Further, there is a long felt need in the art for a CPAP bracket device that allows users to keep their mask easily accessible and put it on without having to ask another person for assistance. Moreover, there is a long felt need in the art for a device that offers a way to dry out the mask and headgear after washing it to prevent the development of mold and bacteria. Further, there is a long felt need in the art for a CPAP bracket handle device that is comprised of aluminum. Finally, there is a long felt need in the art for a CPAP bracket device that retains the headgear and mask in such a way that the user can slide their head toward the mount and apply their headgear with ease.

The present invention, in one exemplary embodiment, is a novel CPAP bracket device. The device allows elderly, handicapped, or other physically limited individuals to put on a CPAP mask without assistance. The device is comprised of an aluminum bracket component that may be mounted to a wall. The bracket component includes two bent aluminum rods which are used to hold the CPAP headgear and mask. The headgear and mask are held in such a way that the user can slide their head toward the mount and apply their headgear with ease. The mount also offers a secure storage place for the mask and headgear to prevent it from being misplaced, or to conveniently dry it after it has been washed. The present invention also includes a novel method of supporting CPAP headgear and mask to keep the mask easily accessible. The method includes the steps of providing a bracket component that may be mounted to a wall. The method also comprises mounting the bracket component to a wall. Further, the method comprises positioning the mask and headgear on the bracket component. The method also comprises sliding a user's head toward the mask and headgear, such that the mask is applied with ease. Finally, the method comprises preventing the mask and headgear from being misplaced, or to conveniently dry it after it has been washed.

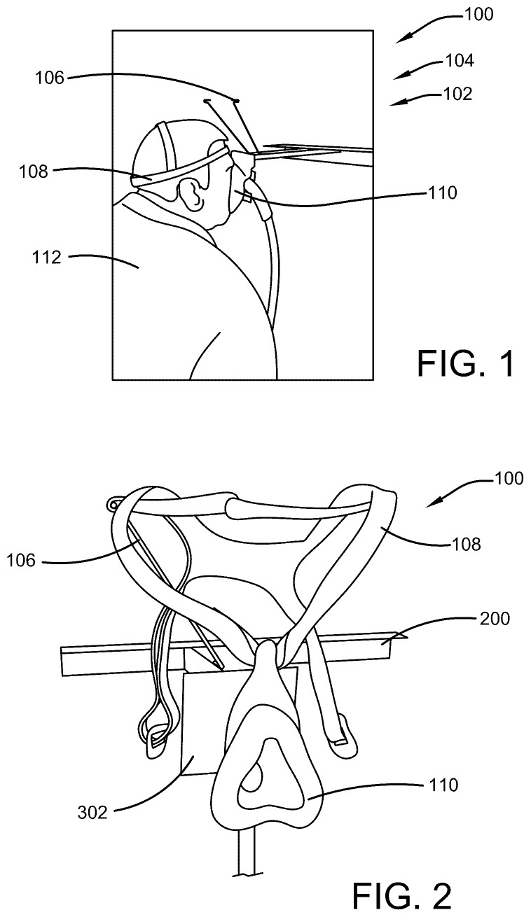

Referring initially to the drawings, FIG. 1 illustrates a perspective view of one embodiment of the CPAP bracket device 100 of the present invention. In the present embodiment, the CPAP bracket device 100 is an improved CPAP bracket device 100 that provides a user 112 with a bracket for supporting CPAP headgear 108 and its mask 110. Specifically, the CPAP bracket device 100 comprises a bracket component 102 which is mounted to a wall 104. The bracket component 102 includes two bent rods 106 which are used to hold the CPAP headgear 108 and mask 110. The device 100 offers a secure storage place for the mask 110 and headgear 108 to prevent it from being misplaced.

Generally, the CPAP bracket device 100 offers a secure storage place for CPAP mask 110 and headgear 108. Thus, the device 100 allows users 112 to keep their mask 110 and headgear 108 easily accessible and be able to put it on without having to ask another person for assistance. Further, the CPAP bracket device 100 also offers a way to dry out the CPAP mask 110 and headgear 108 after washing it to prevent the development of mold and bacteria.

As shown in FIG. 2, the CPAP bracket device 100 comprises a bracket component 102 that may be mounted to a wall 104 or other vertical support surface, as needed. The bracket component 102 comprises an L-shaped support component 200 that is secured directly to the wall 104 or other vertical support surface. The L-shaped support component 200 can be any suitable size and length as is known in the art, as long as the L-shaped support component 200 is of a size and length to support a standard CPAP mask 110 and headgear 108 with ease. Further, the L-shaped support component 200 comprises a front surface 202 which faces out from the wall 104 and a rear surface 204 which secures to the wall 104. Typically, the L-shaped support component 200 is secured to the wall 104 via wall anchors, screws, nails, bolts, etc., or any other suitable fastening means as is known in the art.

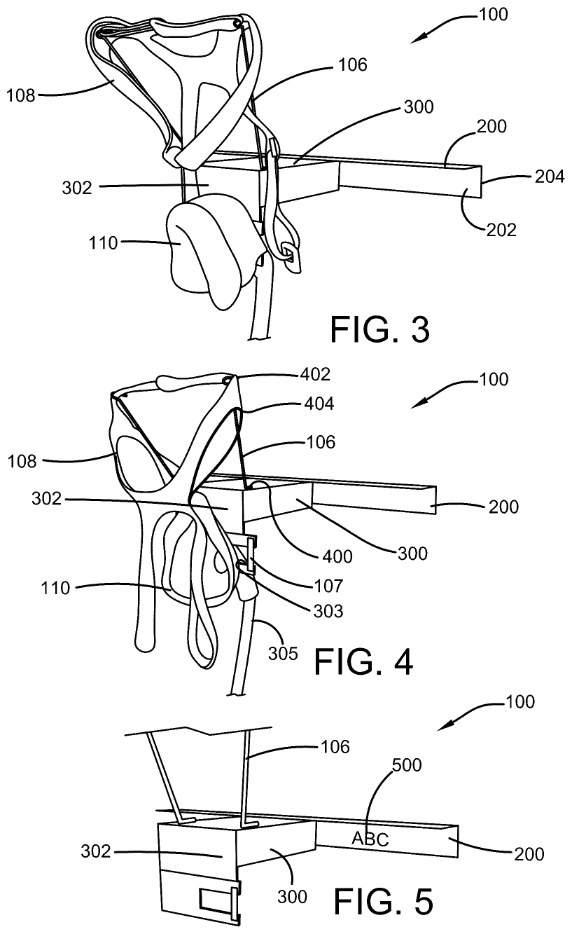

As shown in FIG. 3, a pair of posts 300 are secured near the middle of the front surface 202 of the L-shaped support component 200 via screws, nails, bolts, etc., or any suitable fastening means as is known in the art. The pair of posts 300 are positioned perpendicular to the L-shaped support component 200. The pair of posts 300 are secured with a front plate 302 at the front to keep the pair of posts 300 secured together during use.

As shown in FIG. 4, the bracket component 102 includes two bent rods 106 which are used to hold the CPAP headgear 108 and mask 110. The two bent rods 106 are secured to a top 400 of the pair of posts 300 and angle outwards and upwards. The two bent rods 106 are secured via screws, nails, bolts, etc., or any other suitable fastening means as is known in the art. The two bent rods 106 are angled in such a fashion that CPAP mask 110 and headgear 108 can be hung from the rods 106. Further, the ends 402 of the two bent rods 106 can comprise eyelets 404 or other openings, which parts of the headgear 108 or mask 110 can be secured to.

Further, the CPAP bracket device 100 allows elderly, handicapped, or other physically limited individuals to put on a CPAP mask 110 without assistance. Specifically, the two bent rods 106 are angled, such that the CPAP headgear 108 and mask 110 are held in such a way that the user 112 can slide their head toward the mount and apply their headgear 108 with ease. Thus, the CPAP bracket device 100 allows users 112 to keep their mask 110 easily accessible and put it on without having to ask another person for assistance.

In one embodiment, a simple locking mechanism 107 is incorporated into the bottom of the front plate 302, wherein a slot 303 is shown for retaining a CPAP hose 305. The simple locking mechanism retains the CPAP hose 305 in place and prevent it from accidentally sliding out of the device 100 prematurely before the CPAP headgear 108 and mask 110 are completely secured in place.

In another embodiment, the device 100 can be available in different widths to accommodate all major mask manufacturers. In yet another embodiment, the device 100 would have a one size that fits most, as most CPAP headgear 108 and masks 110 have a swivel connection at the mask 110.

Accordingly, the CPAP bracket device 100 also offers a secure storage place for the mask 110 and headgear 108 to prevent it from being misplaced, or to conveniently dry it after it has been washed to prevent the development of mold and bacteria.

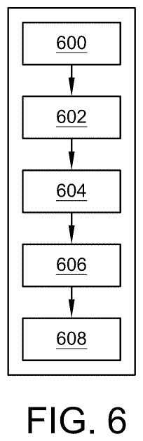

As shown in FIG. 5, the CPAP bracket device 100 is manufactured from aluminum, stainless steel, plastic, such as polycarbonate, or other suitable materials as is known in the art, or a combination of such materials.

In yet another embodiment, the CPAP bracket device 100 comprises a plurality of indicia 500. The bracket component 102 of the device 100 may include advertising, a trademark, or other letters, designs, or characters, printed, painted, stamped, or integrated into the bracket component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be bracket, CPAP headgear, or brand related.

FIG. 6 illustrates a flowchart of the method of supporting CPAP headgear and mask to keep the mask easily accessible. The method includes the steps of at 600, providing a bracket component that may be mounted to a wall. The method also comprises at 602, mounting the bracket component to a wall. Further, the method comprises at 604, positioning the mask and headgear on the bracket component. The method also comprises at 606, sliding a user's head toward the mask and headgear, such that the mask is applied with ease. Finally, the method comprises at 608, preventing the mask and headgear from being misplaced, or to conveniently dry it after it has been washed.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “CPAP bracket device”, “CPAP device”, “bracket device”, and “device” are interchangeable and refer to the CPAP bracket device 100 of the present invention.

Notwithstanding the forgoing, the CPAP bracket device 100 of the present invention can be of any suitable size and configuration as is known in the art without affecting the overall concept of the invention, provided that it accomplishes the above stated objectives. One of ordinary skill in the art will appreciate that the CPAP bracket device 100 as shown in FIGS. 1-6 is for illustrative purposes only, and that many other sizes and shapes of the CPAP bracket device 100 are well within the scope of the present disclosure. Although the dimensions of the CPAP bracket device 100 are important design parameters for user convenience, the CPAP bracket device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A CPAP bracket device that provides a user with a bracket for supporting CPAP headgear and mask, the CPAP bracket device comprising:
a bracket component;
wherein the bracket component is mounted to a wall; and
further wherein the bracket component comprises two bent rods which are used to hold the CPAP headgear and mask.

2. The CPAP bracket device of claim 1, wherein the bracket component comprises an L-shaped support component that is secured directly to the wall.

3. The CPAP bracket device of claim 2, wherein the L-shaped support component comprises a front surface which faces out from the wall and a rear surface which secures to the wall.

4. The CPAP bracket device of claim 3, wherein the L-shaped support component is secured to the wall via wall anchors, screws, nails, or bolts.

5. The CPAP bracket device of claim 4, wherein a pair of posts are secured near a middle of the front surface of the L-shaped support component.

6. The CPAP bracket device of claim 5, wherein the pair of posts are positioned perpendicular to the L-shaped support component.

7. The CPAP bracket device of claim 6, wherein the pair of posts are secured with a front plate at a front to keep the pair of posts secured together during use.

8. The CPAP bracket device of claim 7, wherein the two bent rods are secured to a top of the pair of posts and angle outwards and upwards.

9. The CPAP bracket device of claim 8, wherein the two bent rods are angled in such a fashion that CPAP mask and headgear can be hung from the two bent rods.

10. The CPAP bracket device of claim 9, wherein ends of the two bent rods comprise eyelets which allow parts of the CPAP mask and headgear to be secured to.

11. The CPAP bracket device of claim 10, wherein the CPAP bracket device allows elderly, handicapped, or other physically limited individuals to put on a CPAP mask and headgear without assistance.

12. The CPAP bracket device of claim 11, wherein the two bent rods are angled, such that the CPAP headgear and mask are held in such a way that a user can slide their head toward the CPAP headgear and mask and apply it with ease.

13. A CPAP bracket device that provides a user with a bracket for supporting CPAP headgear and mask, the CPAP bracket device comprising:
a bracket component comprising an L-shaped support component that is secured directly to a wall; and
wherein the L-shaped support component comprises a front surface which faces out from the wall and a rear surface which secures to the wall;
wherein a pair of posts are secured near a middle of the front surface of the L-shaped support component and positioned perpendicular to the L-shaped support component;
wherein the bracket component comprises two bent rods which angle outwards and upwards to hold the CPAP headgear and mask; and
further wherein the CPAP bracket device allows elderly, handicapped, or other physically limited individuals to put on a CPAP mask and headgear without assistance.

14. The CPAP bracket device of claim 13, wherein the bracket component allows the CPAP headgear and mask to be stored and dried after washing to prevent development of mold and bacteria.

15. The CPAP bracket device of claim 13, wherein the pair of posts are secured with a front plate at a front to keep the pair of posts secured together during use.

16. The CPAP bracket device of claim 13, wherein ends of the two bent rods comprise eyelets which allow parts of the CPAP mask and headgear to be secured to.

17. The CPAP bracket device of claim 15, wherein a simple locking mechanism is incorporated into a bottom of the front plate, wherein a slot is shown for retaining a CPAP hose to retain the CPAP hose in place and prevent it from accidentally sliding out prematurely before the CPAP mask and headgear are completely secured in place.

18. The CPAP bracket device of claim 13, wherein the CPAP bracket device is manufactured from aluminum, stainless steel, plastic, such as polycarbonate, or a combination of such materials.

19. The CPAP bracket device of claim 13 wherein the CPAP bracket device is manufactured in various widths.

20. A method of supporting CPAP headgear and mask to keep the mask easily accessible, the method comprising the following steps:
providing a bracket component that may be mounted to a wall;
mounting the bracket component to a wall;
positioning the mask and headgear on the bracket component;
sliding a user's head toward the mask and headgear, such that the mask is applied with ease; and
preventing the mask and headgear from being misplaced, or to conveniently dry it after it has been washed.
